(19) United States
(12) Patent Application Publication
(10) Pub. No.: US 2007/0178466 A1
(43) Pub. Date: Aug. 2, 2007

(54) Composition for deaminating dna and method of detecting methylated dna

(75) Inventors: Hikoya Hayatsu, Okayama (JP); Kazuo Negishi, Okayama (JP); Masahiko Shiraishi, Chiba (JP)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Toyo Boseki Kabushiki Kaisha, Osaka (JP)

(21) Appl. No.: 10/593,130

(22) PCT Filed: Nov. 30, 2004
(86) PCT No.: PCT/JP04/17782

§ 371 Date: Sep. 18, 2006

(30) Foreign Application Priority Data
Apr. 8, 2004 (JP) 2004-114476

(51) Int. Cl. C12Q 1/68; C07H 21/02; C12N 1/08

(52) U.S. Cl. 435/6; 435/270; 536/25.1

(57) ABSTRACT

(1) A sulfite composition having a sulfite concentration of more than 6.2 M, (2) a method for deaminating DNA using a sulfite composition described in (1), (3) a method for detecting methylated DNA using a sulfite composition described in (1), (4) a kit for deaminating DNA or for detecting methylated DNA comprising a sulfite composition described in (1).

### TECHNICAL FIELD

The present invention relates to a composition for deaminating DNA and a method for deaminating DNA. Further, it relates to a method for detecting methylated DNA in a sample.

### BACKGROUND ART

It is known that methylation of genomic DNA regulates the expression of genes in a eukaryote. Therefore, by detecting methylated DNA, important genetic information can be obtained.

In particular, 5-methylcytosine is only a physiologically modified base present in the genome of a eukaryote, and it is also known that aberration of DNA methylation causes a genetic disease or a cancer. Accordingly, it is particularly important to detect the methylation status of cytosine of a specific nucleotide sequence in the genome.

However, 5-methylcytosine forms a complementary base pair with guanine in the same manner as cytosine, therefore, it is extremely difficult to detect it by sequence determination or PCR as it is.

The method that is used most frequently as a means for solving this problem is a method for deaminating cytosine by reacting genomic DNA with a sulfite, and converting it to uracil by alkaline hydrolysis. 5-methylcytosine has a very low reactivity with this reagent (see, for example, Hayatsu et al., Biochemistry, Vol. 9, pp. 2858-2865 (1970)). Therefore, if nucleotide sequence is determined after such a treatment is performed, cytosine will be determined as thymine, and only the location of 5-methylcytosine wilT be determined as cytosine, whereby it will be possible to identify the location of 5-methylcytosine (see, for example, Formmer et al., Proc. Natl. Acad. Sci. USA, Vol. 89, pp. 1827-1831 (1992)).

Here, the reaction conditions of DNA with a sulfite are generally set at 50° C. for 12 to 16 hours in 4.9 M sodium bisulfite solution (pH 5) (see, for example, Eads et al., Methods in Molecular Biology, Vol. 200, pp. 71-85 (2002)). However, such a prolonged reaction became one of the causes why detection of methylated cytosine cannot be rapidly carried out.

On the other hand, as life science-related industries or bio-related industries have made progress recently, data processing of enormous amount of DNA-related information or rapid acquisition of genetic information has been demanded.

Accordingly, the development of a method for rapidly deaminating DNA and rapidly detecting methylated DNA was needed.

### DISCLOSURE OF THE INVENTION

A main object of the present invention is to provide a method for rapidly performing deamination reaction of DNA and detecting methylated DNA in a sample in a short time. More particularly, it is to provide a method for rapidly performing deamination reaction of cytosine and detecting methylated cytosine in a sample in a short time.

In order to attain the objects described above, the present inventors conducted intensive investigations. As a result, they found that by reacting DNA with a sulfite solution with a high sulfite concentration, deamination reaction of cytosine proceeds in an extremely short time. They further conducted investigations, thus the present invention has been accomplished.

In other words, the present invention relates to a sulfite composition, a method for deaminating DNA, a method for detecting methylated DNA and a kit for deaminating DNA or for detecting methylated DNA described below.

Item 1: A sulfite composition having a sulfite concentration of more than 6.2 M.

To be more specific, it is a sulfite composition having a sulfite concentration of more than 6.2 M for deaminating DNA or for detecting methylated DNA. In other words, it is an invention relates to use of a sulfite composition having a sulfite concentration of more than 6.2 M for deaminating DNA or for detecting methylated DNA.

Item 2: The sulfite composition described in the item 1 having a sulfite concentration of more than 6.2 M and 10 M or less.

Item 3: The sulfite composition described in the item 1 or 2 having a pH of 5.0 to 5.6.

Item 4: The sulfite composition described in any one of the items 1 to 3 comprising 2 types or more of sulfites.

Item 5: The sulfite composition described in any one of the items 1 to 4 comprising 2 types or more of sulfites selected from the group consisting of ammonium salts and sodium salts of sulfites.

Item 6: The sulfite composition described in any one of the items 1 to 5 comprising ammonium sulfite, ammonium bisulfite and sodium bisulfite.

Item 7: A method for deaminating DNA comprising the following steps of:

(1) treating a sample containing a single-stranded DNA with a sulfite composition having a sulfite concentration of more than 6.2 M; and

(2) treating the sample treated in (1) with an alkali.

To be more specific, the sulfite composition in the step (1) is a sulfite composition described in any one of the items 1 to 6.

Item 8: The method for deaminating DNA described in the item 7 comprising the following step (0) before the step (1):

(0) denaturing a double-stranded DNA in the sample into single-stranded DNAs.

Item 9: The method for deaminating DNA described in any one of the items 7 to 8, wherein the DNA in the step (1) is DNA comprises cytosine.

Item 10: The method for deaminating DNA described in any one of the items 7 to 9, wherein the sulfite composition in the step (1) is a sulfite composition having a sulfite concentration of more than 6.2 M and 10 M or less.

Item 11: The method for deaminating DNA described in any one of the items 7 to 10, wherein the step (1) is a step of performing the treatment in a pH range of about 5 to 5.6.

Item 12: The method for deaminating DNA described in any one of the items 7 to 11, wherein the step (1) is a step of performing the treatment at a temperature of about 60 to 95° C. for about 5 to 60 minutes.

Preferably, it is a method for deaminating DNA described in any one of the items 7 to 11, wherein the step (1) is a step of performing the treatment at a temperature of about 70 to 90° C. for about 5 to 60 minutes.

Item 13: A method for detecting methylated DNA comprising the following steps of:

(a) performing deamination treatment by treating a sample containing a single-stranded DNA with a sulfite composition having a sulfite concentration of more than 6.2 M and treating it with an alkali; and

(b) detecting methylated DNA in the sample obtained in (a).

To be more specific, the step (a) is a step of performing deamination treatment by a method described in any one of the items 7 to 12.

Item 14: The method for detecting methylated DNA described in the item 13, wherein the DNA in the step (a) is DNA comprises cytosine, and the step (b) is a step of detecting methylated cytosine in the sample obtained in (a).

Item 15: The method for detecting methylated DNA described in the item 14, wherein the step (b) is a step of, detecting methylated cytosine in the sample by using any means of nucleotide sequence determination, a DNA chip and a restriction enzyme.

To be more specific, the step (b) is a step of detecting methylated cytosine in the sample by using any means of (i) identifying the locations of cytosine and thymine by nucleotide sequence determination after amplifying DNA in the sample by PCR, (ii) identifying cytosine and thymine by using a DNA chip in which a probe hybridizing to DNA in the case where cytosine is converted to thymine and a probe hybridizing to DNA in the case where cytosine is not converted to thymine have been immobilized after amplifying DNA in the sample by PCR, or (iii) determining cytosine and thymine based on the presence or absence of a DNA fragment by using a restriction enzyme which digests DNA and/or a restriction enzyme which does not digest DNA by converting cytosine to thymine after amplifying DNA in the sample by PCR.

Item 16: The method for detecting methylated DNA described in the item 14, wherein the step (b) is a step of detecting methylated cytosine by means of amplifying DNA in the sample using at least one primer that can amplify a nucleic acid in the case where cytosine in the sample DNA is converted to uracil and at least one primer that can amplify a nucleic acid in the case where cytosine is not converted to uracil, and identifying the locations of 5-methylcytosine and uracil based on the presence or absence of amplification.

Item 17: A kit for deaminating DNA comprising a sulfite composition described in the item 1.

To be more specific, it is a kit for deaminating DNA comprising a sulfite composition described in any one of the items 1 to 6.

Preferably, it is a kit further comprising a means of detecting DNA, or a kit for deaminating DNA further comprising a primer for amplifying DNA.

Item 18: A kit for detecting methylated DNA comprising a sulfite composition described in the item 1.

To be more specific, it is a kit for detecting methylated DNA comprising a sulfite composition described in any one of the items 1 to 6.

Preferably, it is a kit for detecting methylated DNA further comprising a means of detecting DNA, or a kit for methylated DNA further comprising a primer for amplifying DNA.

### MODE FOR CARRYING OUT THE INVENTION

Hereunder, the present invention will be described in detail.

Sulfite Composition

One of the aspects of the present invention is a sulfite composition showing a high sulfite concentration.

The sulfurous acid in the present invention includes H₂SO₃, HSO₃⁻, SO₃⁻⁻ (represented by a chemical formula) and the like. Under an acidic condition, which is a preferred embodiment of the present invention, almost all are present as a bisulfite ion (HSO₃⁻).

The sulfite concentration in the sulfite composition of the present invention is more than 6.2 M, preferably 8 M or more. In addition, it is preferably 10 M or less. If the concentration is too low, there is a tendency that the reaction rate of DNA deamination will decrease. On the other hand, if the concentration is too high, a crystal will be easily formed.

It is preferred that the pH of the sulfite composition of the present invention is substantially the same as the optimal pH for deamination reaction of DNA. Therefore, the pH of the sulfite composition of the present invention ranges preferably from about 4.0 to 6.0, more preferably from about 5.0 to 5.6.

Accordingly, a most preferred aspect of the sulfite composition of the present invention is the case where the sulfite concentration is 8 M or more and 10 M or less, and the pH is from 5.0 to 5.6.

It is preferred that such a sulfite composition of the present invention having a high sulfite concentration contains 2 or more types of sulfites.

Examples of the types of sulfites include sodium salts, ammonium salts and potassium salts of sulfites and the like.

Specific examples include sodium bisulfite (NaHSO₃), sodium sulfite (Na₂SO₃), ammonium sulfite ((NH₄)₂SO₃), ammonium bisulfite ((NH₄)HSO₃), potassium sulfite (K₂SO₃) and the like.

Among these, for a reason related to solubility and preparation of pH, it is preferred to use 2 or more types of sulfites in combination selected from the group consisting of sodium salts of sulfites and ammonium salts of sulfites.

Further, it is preferred to use ammonium bisulfite, ammonium sulfite and sodium bisulfite in combination.

A preparation method of the sulfite composition of the present invention is not particularly limited. However, in the case of combination of ammonium bisulfite, ammonium sulfite and sodium bisulfite, it is preferred that powders of sodium bisulfite and ammonium sulfite are added to a solution of ammonium bisulfite, and the mixture is heated for about 5 minutes to 40 minutes, more preferably for about 10 to 20 minutes, at 50 to 95° C., preferably at 70 to 90° C.

The sulfite composition of the present invention is preferably used for deaminating DNA or for detecting methylated DNA.

Method for Deaminating DNA

One of the aspects of the present invention is a method for deaminating DNA.

A method for deaminating DNA of the present invention comprises (1) a step of treating a sample containing a single-stranded DNA with the sulfite composition of the present invention described above; and (2) a step of treating the sample treated in (1) with an alkali.

In the case where the sample contains a double-stranded DNA, a step of denaturing the double-stranded DNA in the sample into single-stranded DNAs may be further included before the step (1).

Further, in the case of treating high molecular weight DNA, for example, genomic DNA, a step of digesting and fragmenting DNA with a restriction enzyme before the step of denaturation of DNA may be added as needed.

As the method of denaturing a double-stranded DNA into single-stranded DNAs, for example, a heat treatment, an alkali treatment and the like may be exemplified. A condition of the heat treatment is not particularly limited, however, the treatment is carried out, for example, at about 95 to 100° C. for about 5 minutes to 10 minutes. A condition of the alkali treatment is not particularly limited, either, however, the treatment is carried out, for example, with an alkali at a concentration of 0.2 N or more for about 20 minutes to 60 minutes at about 30° C. to 42° C. In particular, a method of performing the treatment with sodium hydroxide at a concentration of about 0.3 N for about 30 minutes at about 30 to 37° C. is preferred.

In the step (1), it is preferred to use a sulfite composition having a sulfite concentration of more than 6.2 M, preferably 8 M or more, and 10 M or less. If the concentration is too low, the reaction rate of DNA deamination will decrease. On the other hand, if the concentration is too high, a crystal will be easily formed.

In addition, it is preferred that the treatment of a sample with the sulfite composition is carried out in a pH range of about 5.0 to 5.6. Either a too low or too high pH will cause the deamination ratio to decrease.

It is preferred that the treatment temperature is from about 60 to 95° C., more preferably from about 70° C. to 90° C. If the temperature is too low, sulfite will be crystallized, whereby the reaction will be difficult to proceed. In addition, if the temperature is too high, degradation of DNA will rapidly proceed, whereby there is a possibility that a following analysis may have some difficulty.

It is preferred that the treatment time is about 5 minutes to 60 minutes. If the time is too short, deamination will be insufficient. On the other hand, if the time is too long, damage of the sample such as degradation of DNA will be likely to occur.

There is a tendency that the step (1) proceeds more rapidly as the sulfite concentration increases. Accordingly, it is preferred to avoid adding an unnecessary solution other than a sample and the sulfite composition wherever possible in the step (1).

The alkali treatment in the step (2) is not particularly limited as long as it is a treatment capable of detaching a sulfite group bound to a nucleic acid. For example, a method of adding sodium hydroxide, potassium hydroxide, ammonia and/or Tris and the like to a sample and treating the sample at a pH of 9.0 or more for about 10 minutes to 120 minutes can be exemplified. In particular, it is preferred that sodium hydroxide at a concentration of about 0.2 N is added to a sample and the sample is treated for about 10 minutes.

The type of the sample to be targeted of the present invention is not particularly limited, and a variety of cells including blood, cancer cells, cultured cells and the like or tissues can be applied. The type of DNA is not limited, and for example, plasmid DNAs, genomic DNAs and the like can be applied. The origine of DNA is not particularly limited, and for example, a variety of animals including human and mouse, yeast, bacteria and the like can be applied.

The method for deaminating DNA of the present invention is preferably used particularly for deamination of DNA comprising cytosine. Specifically, the method can be used as a method for deaminating DNA comprising (1) a step of treating a sample containing a single-stranded DNA comprising cytosine with the sulfite composition of the present invention described above, and (2) a step of converting cytosine to uracil by treating the sample treated in (1) with an alkali.

Method for Detecting Methylated DNA

One of the aspects of the present invention is a method for detecting methylated DNA.

A method for detecting methylated DNA of the present invention comprises the following steps.

(a) A step of performing deamination treatment by treating a sample containing a single-stranded DNA with a sulfite composition having a sulfite concentration of more than 6.2 M and treating it with an alkali.

(b) A step of detecting methylated DNA in the sample obtained in (a).

Specifically, the step (a) is a step of deaminating DNA in accordance with the method for deaminating DNA of the present invention described above. The sulfite concentration in the sulfite composition is preferably 8 M or more. In addition, it is preferably 10 M or less. Further, it is preferred that the treatment with the sulfite composition is carried out in a pH range of about 5 to 5.6. In addition, the treatment temperature is preferably 60 to 95° C., and is further preferably 70 to 90° C. In addition, the treatment time is preferably about 10 to 60 minutes.

In addition, in the step (a), further a treatment of denaturing a double-stranded DNA in the sample into single-stranded DNAs may be performed. Further, in the case of treating high molecular weight DNA, for example, genomic DNA, a step of digesting and fragmenting DNA with a restriction enzyme before the step of denaturation of DNA may be added as needed.

The detection method of the present invention is preferably used for detecting particularly methylated cytosine among methylated DNAs. Specifically, it can be used as a method comprising (a) a step of deaminating DNA by treating a sample containing a single-stranded DNA comprising cytosine with the sulfite composition of the present invention and treating the sample with an alkali, whereby cytosine in the DNA is converted to uracil, and (b) a step of detecting methylated cytosine in the sample treated in (a).

In the step (b), the detection of methylated cytosine can be performed, for example, by means of using nucleotide sequence determination, a DNA chip or a restriction enzyme.

Specifically, the means of using nucleotide sequence determination is (i) a means of identifying the locations of cytosine and thymine by nucleotide sequence determination after amplifying DNA in the sample by PCR. The means of using a DNA chip is (ii) a means of identifying cytosine and thymine by using a DNA chip in which a probe hybridizing to DNA in the case where cytosine is converted to thymine and a probe hybridizing to DNA in the case where cytosine is not converted to thymine have been immobilized after amplifying DNA in the sample by PCR. In addition, the means of using a restriction enzyme is (iii) a means of determining cytosine and thymine based on the presence or absence of a DNA fragment by using a restriction enzyme which digests DNA and/or a restriction enzyme which does not digest DNA by converting cytosine to thymine after amplifying DNA in the sample by PCR.

In addition, in the step (b), detection of methylated cytosine may be carried out by using a means of determining cytosine and thymine based on the presence or absence of amplification by subjecting a DNA sample to amplification reaction using at least one primer that can amplify a nucleic acid in the case where cytosine in the DNA sample is converted to uracil and at least one primer that can amplify a nucleic acid in the case where cytosine is not converted to uracil, respectively.

In any means, a method including a DNA amplification method such as PCR is preferred.

Kit

One of the aspects of the present invention is a kit for deaminating DNA or a kit for detecting methylated DNA.

The kit of the present invention is characterized by comprising the sulfite composition of the present invention described above.

In the kit of the present invention, an appropriate means for deaminating DNA or for detecting methylated DNA, a means for purifying DNA, a means for labeling, a reagent or the like can be included as needed. In addition, aprimer for amplifying DNA that can be used for PCR or the like can be included.

Examples of the detection means may include a variety of primers, probes, restriction enzymes, fluorescent dyes, and/or a variety of media and the like.

The kit of the present invention can be particularly preferably used in implementing the method for deaminating DNA and the method for detecting methylated DNA of the present invention described above.

### ADVANTAGE OF THE INVENTION

By using the sulfite composition with a high sulfite concentration of the present invention, a treatment of deaminating DNA can be carried out in a short time.

Conventionally, it took long time (about 12 to 16 hours) for deamination treatment of DNA, and it was difficult to rapidly perform detection of methylated DNA. However, according to the present invention, deamination of DNA can be performed in a short time, and further, it becomes possible to rapidly perform detection of methylated DNA.

In particular, according to the present invention, it becomes possible to perform conversion of cytosine to uracil in a short time, and further, it becomes possible to rapidly perform detection of methylated cytosine.

The present invention can be utilized in various techniques such as acquisition of genetic information and development of a DNA-related technique. For example, it has been reported that aberration of methylated DNA is associated with various diseases such as a cancer, however, by rapidly detecting methylated DNA according to the present invention, the efficiency of diagnosis, a gene test or the like is considerably increased. In addition, the present invention is also useful as a tool for studying methylated DNA.

In this way, the present invention largely contributes to promoting life science industries including medical services or bio-related industries.

### BRIEF DESCRIPTION OF THE DRAWINGS

FIG. 1 is a graph showing the deamination ratio in a sample treated with a sulfite composition as the remaining amount of cytosine. Closed circles (●) indicate the case where 2′-deoxycytidine was treated with 9 M sodium bisulfite-ammonium solution at 70° C. Open lozenges (⋄) indicate the case where 2′-deoxycytidine was treated with 5.3 M sodium bisulfite solution at 70° C. Open squares (□) indicate the case where 5-methyl-2′-deoxycytidine was treated with 9 M sodium bisulfite-ammonium solution at 70° C. Closed triangles (▴) indicate the case where 5-methyl-2′-deoxycytidine was treated with 9 M sodium bisulfite-ammonium solution at 90° C.

FIG. 2 is a graph showing the pH dependency of deamination reaction of DNA.

FIG. 3 shows graphs showing the results of analyzing a salmon testis DNA sample by HPLC. FIG. 3a shows the results of analyzing a sample treated with a sulfite composition of the present invention and FIG. 3b shows the results of analyzing an untreated sample. In FIG. 3, C indicates 2′-deoxycytidine, U indicates 2′-deoxyuridine, mC indicates 5-methyl-2′-deoxycytidine, G indicates 2′-deoxyguanosine, T indicates thymidine and A indicates 2′-deoxyadenosine.

FIG. 4 shows views related to the analysis by a sulfite treatment of CDH1 gene in MCF-7 cell. FIG. 4(A) shows the amplified genomic region. FIG. 4(B) shows the sequence of the amplified region. The bold characters indicate CpG dinucleotides. FIG. 4(C) shows the results of PCR amplification when genomic DNA subjected to a sulfite treatment was serially diluted. The samples in a were treated by a conventional method (with a sulfite composition having a sulfite concentration of 3.6 M at 55° C. for 20 hours). The samples in b were treated with a sulfite composition of the present invention at 90° C. for 20 minutes. The samples in c were treated with a sulfite composition of the present invention at 70° C. for 40 minutes. Five hundred nanograms (lane 1), 50 ng (lane 2), 5 ng (lane 3), 500 pg (lane 4) and 50 pg (lane 5) of DNA was used as a template. FIG. 4 (D) shows the results of analyzing the nucleotide sequences of plasmid clones. Each row indicates an independent plasmid clone. Open circles (◯) and closed circles (●) indicate thymine and cytosine, respectively. The dotted circle at the position 2 is not counted because this position was heterozygous in the MCF-7 cells. The arrows indicate the positions of the cytosine in CpG nucleotide.

FIG. 5 shows views related to the analysis by a sulfite treatment of RASSF1A gene in MCF-7 cell. FIG. 5(A) shows the amplified genomic region. FIG. 5(B) shows the sequence of the amplified region. The bold characters indicate CpG dinucleotides. Since a complementary strand was used as a template, the position of a methylated cytosine of the complementary strand is indicated as a guanine residue. FIG. 5(C) shows the results of PCR amplification when genomic DNA subjected to a sulfite treatment was serially diluted. The samples in a were treated by a conventional method (with a sulfite composition having a sulfite concentration of 3.6 M at 55° C. for 20 hours). The samples in b were treated with a sulfite composition of the present invention at 90° C. for 20 minutes. The samples in c were treated with a sulfite composition of the present invention at 70° C. for 40 minutes. Five hundred nanograms (lane 1), 50 ng (lane 2), 5 ng (lane 3), 500 pg (lane 4) and 50 pg (lane 5) of DNA was used as a template. FIG. 5(D) shows the results of analyzing the nucleotide sequences of plasmid clones. Each row indicates an independent plasmid clone. Open circles (◯) and closed circles (●) indicate thymine and cytosine, respectively. The arrows indicate the positions of the cytosine in CpG nucleotide.

### BEST MODE FOR CARRYING OUT THE INVENTION

Hereunder, the present invention will be described in more detail with reference to Examples and Experimental Examples, however, the present invention is not limited to the following Examples.

[Method for Measurement]

0-A. Measurement of Sulfite Concentration

The measurement of sulfite concentration was performed by utilizing the fact that sulfur dioxide is generated from a sulfite in a solution of hydrochloric acid and the absorbance at 276 nm (A₂₇₆) changes depending on the amount of generated sulfur dioxide.

To a cuvette for measuring absorbance (1×1×4 cm, manufactured by Hitachi High-Technologies Co.), 3 ml of 0.1 N hydrochloric acid (manufactured by Wako Pure Chemical Co., Ltd.) was added. To the cuvette, 30 μl of a sulfite solution diluted with distilled water was added, the cuvette was covered with parafilm, and inverted 3 times to mix the solutions. Then, the absorbance at 276 nm was measured with a spectrophotometer (Model U-2800, manufactured by Hitachi Instruments Service Co., Ltd).

The solutions of sodium sulfite (manufactured by Wako Pure Chemical Co., Ltd.) diluted from 0.2 mM to 3 mM were used as standard solutions and the absorbance thereof was measured in the same way, whereby the sulfite concentration in a sample was calculated from the absorbance values of the standard solutions and the sample.

When the sulfite concentration of a commercially available 50% ammonium bisulfite (manufactured by Wako Pure Chemical Co., Ltd.) was measured by this method, it was from 6.0 M to 6.2 M.

0-B Measurement of Solubility

The solubilities of sodium bisulfite, sodium sulfite and ammonium sulfite monohydrate (all manufactured by Wako Pure Chemical Co., Ltd.) were measured as follows.

At 30° C. or 70° C., a solution was prepared by adding sodium bisulfite, sodium sulfite or ammonium sulfite monohydrate to 10 ml of distilled water until no more dissolved. Then, the mass, volume and pH at that time were measured. In addition, with regard to each solution, the sulfite concentration was measured in accordance with the method 0-A described above.

In Table 1, the measured values and the concentrations calculated from the measured values are shown.

With regard to the concentration in the table, the calculated value represents the sulfite concentration (M (mol/l)) calculated from the mass and the molecular weight of each dissolved sulfite. In addition, the measured value represents the sulfite concentration (M) measured in accordance with the method 0-A described above.

TABLE 1

| | | Concentration | | | |
|---|---|---|---|---|---|
| | | | M | | |
| | | | Calculated | Measured | |
| Reagent | Temperature | g/ml | value | value | pH |
| Sodium bisulfite | 30° C. | 0.49 | 5.2 | 5.0 | 4.4 |
| | 70° C. | 0.61 | 6.5 | 5.9 | 4.5 |
| Sodium sulfite | 30° C. | 0.20 | 1.6 | 1.5 | 10.3 |
| | 70° C. | 0.26 | 2.1 | 2.1 | 10.5 |
| Ammonium sulfite | 30° C. | 0.51 | 3.5 | 3.5 | 8.5 |
| monohydrate | 70° C. | 0.67 | 4.6 | 4.3 | 8.2 |

The solubilities at 70° C. were 5.9 M for sodium bisulfite, 2.1 M for sodium sulfite and 4.3 M for ammonium sulfite monohydrate.

### Example 1

1-A Preparation of High Concentration of Sulfite Solution

To 5.0 ml of 50% ammonium bisulfite solution, 2.08 g of sodium bisulfite and 0.67 g of ammonium sulfite were added and stirred at 70° C. for 5 minutes to dissolve them. The pH of the obtained solution was 5.4 and the sulfite concentration was 10 M. The pH and the sulfite concentration of this solution did not change after the solution was incubated at 70° C. for 4 hours.

Hereinafter the obtained solution is also referred to as a sodium bisulfite-ammonium mixed solution.

1-B. Evaluation of Deamination Reaction Rate of 2′-deoxycytidine and 5-methyl-2′-deoxycytidine

The quantitative determination of deamination reaction product was performed in accordance with the method described in the literature by Sono et al. (Sono et al., J. Am. Chem. Soc., Vol. 96, pp. 4745-4749, (1973)).

A solution in which 2′-deoxycytidine or 5-methyl-2′-deoxycytidine (manufactured by Sigma Co., Ltd.) was dissolved in distilled water to a final concentration of 0.2 M in each case was prepared.

To 25 μl of the 2′-deoxycytidine solution, 250 μl of 5.9 M sodium bisulfite solution prepared in accordance with 0-B (the final reaction concentration: 5.9×250÷(250+25)≈about 5.3 M), or 250 μl of 10 M sodium bisulfite-ammonium solution prepared in Example 1 (the final reaction concentration: 10.0×250÷(250+25)≈about 9.0 M) was added, and a treatment was carried out for 0 to 10 minutes, and 500 μl of chilled water was added to stop the reaction. The reaction solution (75 μl) was mixed with 5 ml of 0.2 M sodium phosphate buffer (pH 7.2) and the mixture was left at room temperature for 40 minutes. Then, the absorbance at 270 nm was measured with a spectrophotometer (Model U-2800, manufactured by Hitachi Instruments Service Co., Ltd).

The absorbance of an unreacted sample (a 2′-deoxycytidine solution with the same concentration, which was not treated with a sulfite solution) was 0.8. The absorbance of only 9 M sodium bisulfite-ammonium solution was 0.05. The absorbance of the unreacted sample was defined as 100%, and the deamination reaction product was quantified by the decrease in the absorbance of a reacted sample.

With regard to 5-methyl-2′-deoxycytidine, the same measurement and quantitative determination were performed as in the case of 2′-deoxycytidine solution described above except for measuring the absorbance at 277 nm.

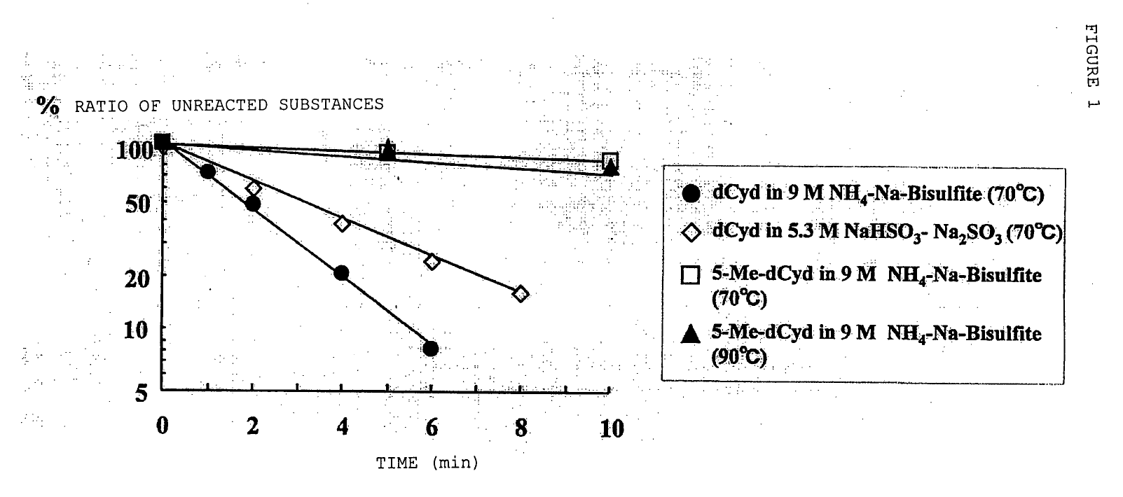

The results of deamination reaction are shown in FIG. 1.

When a treatment was carried out under the condition of 70° C. and pH 5.4, the time taken to convert half of the deoxycytidine to deoxyuridine (t_{1/2}) was 3 minutes in the case of using 5.3 M sodium bisulfite solution. On the other hand, in the case of using 9 M sodium bisulfite-ammonium mixed solution, it was 1.8 minutes.

t_{1/2} (in the case of using a sodium bisulfite solution with a sulfite concentration of 5.3 M)/t_{1/2} (in the case of using a sodium bisulfite-ammonium mixed solution with a sulfite concentration of 9 M) is 1.7, which agrees with the ratio of concentrations (9.0/5.3). In other words, it was indicated that the rate of deamination reaction depends on the sulfite concentration.

In addition, 10 M sodium bisulfite-ammonium solution was serially diluted and a treatment of deamination reaction was carried out at a concentration of 2 M to 9 M. As a result, it was found that the rate of deamination reaction depends on the sulfite concentration.

In addition, in the case where 5-methyl-2′-deoxycytidine was treated with 9 M sodium bisulfite-ammonium mixed solution, the deamination ratio (the ratio of 5-methyl-2-deoxycytidine converted to thymine by deamination) was 16% under the condition of treatment at 70° C. for 10 minutes and 23% under the condition of treatment at 90° C. for 10 minutes.

1-C. Temperature Dependency of Deamination Reaction

The t_{1/2}s of deoxycytidine in the case of performing a treatment with 9 M sodium bisulfite-ammonium solution (pH 5.4) at 90° C., 50° C. and 37° C. were measured by the same method as in 1-B. As a result, they were 1 minute or less, 5 minutes and 17 minutes, respectively.

1-D. Measurement of Time for 100% Deamination

In accordance with the following procedure, the time taken to completely convert 2′-deoxycytidine to 2′-deoxyuridine was measured.

(Method for Measurement)

To 25 μl of 0.2 M 2′-deoxycytidine, 250 μl of 10 M sodium bisulfite-ammonium solution prepared in 1-A (reaction final concentration: 9 M) was added, and a treatment was performed for various times. Then, 500 μl of chilled water was added to stop the reaction. The reaction solution (75 μl) was mixed with 5 ml of 0.2 M sodium phosphate buffer (pH 7.2) and the mixture was left at room temperature for 40 minutes.

Subsequently, after the treatment described above, 10 μl of a sample was subjected to the HPLC analysis described below, and the amounts of 2′-deoxycytidine and 2′-deoxyuridine were measured.

(HPLC Analysis)

Ultrasphere ODS 4.6 mm×25 cm column (manufactured by Beckman-Coulter Co.) was connected to an HPLC analysis system (manufactured by Hitachi Instruments Service Co., Ltd). BufferA (100mM potassium phosphate buffer (pH 7.0)) and Buffer B (90% methanol, 1 mM potassium phosphate buffer (pH 7.0)) were prepared. In the program of the HPLC system, the flow rate was set at 0.7 ml/min, and the buffer concentration profile was set to 100% A: 0 min, 100% A: 5 min, 85% A: 25 min, 55% A: 35 min, and 0% A: 60 min.

The elution times under the condition were 19 minutes for 2′-deoxycytidine, 22 minutes for 2′-deoxyuridine, 25 minutes for 5-methyl-2′-deoxycytidine, 26 minutes for 2′-deoxyguanosine, 28 minutes for thymidine and 32 minutes for 2′-deoxyadenosine. The concentration was calculated from the area of a chart.

(Measurement Results)

From the result of the measurements, it was found that, in the case where deamination treatment was performed by using 9 M sodium bisulfite-ammonium solution (pH 5.4), the time taken to completely (100%) convert 2′-deoxycytidine to 2′-deoxyuridine was 30 minutes at 70° C. and 8 minutes at 90° C.

1-E. pH Dependency of Deamination Reaction

In 50% ammonium bisulfite solution, sodium bisulfite and sodium sulfite were dissolved at a given ratio, and 7 M sulfite solution at a pH of 4.0 to 6.0 was prepared. By using this solution, the deamination ratio of 2-deoxycytidine was measured by the same method as described in 1-B. The reaction time was set to 5 minutes and the temperature was set to 60° C.

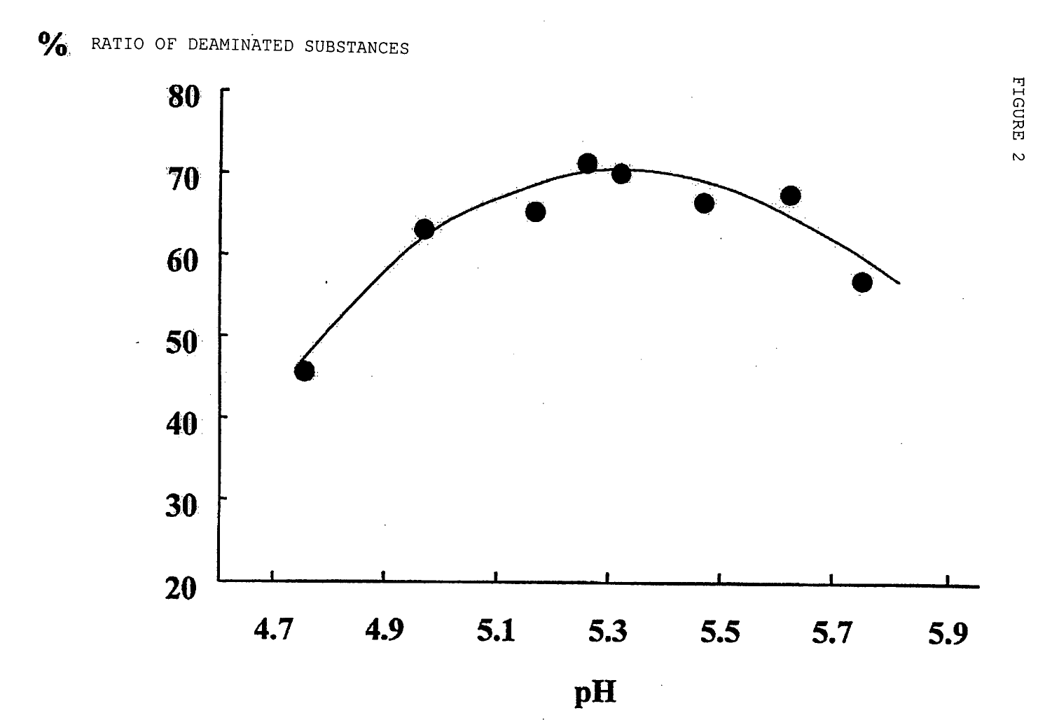

As a result, as shown in FIG. 2, an optimal pH was 5.0 to 5.6.

### Example 2

### Deamination Reaction of Genomic DNA

Salmon testis DNA (manufactured by Sigma Co.) was dissolved in sterile water to a final concentration of 1.6 mg/ml. To 50 μl of this solution, 5 μl of 3 N sodium hydroxide (manufactured by Wako Pure Chemical Co., Ltd.) was added, a treatment was carried out at 30° C. for 30 minutes, whereby a double-stranded DNA was denatured into single-stranded DNAs.

To the obtained solution, 550 μl of 10 M ammonium sulfite-sodium solution (pH 5.4) was added and mixed well. Then, reaction was carried out at 90° C. for 10 minutes (the final concentration of sulfite was 9 M).

Subsequently, the reaction solution was applied to a Sephadex G-50 column (φ15×40 mm, BioRad Econopack 10, manufactured by BioRad Co.), which had been buffered with TE buffer (10 mM Tris-HCl (pH 8), 1 mM EDTA), and a desalting operation was carried out. A DNA fraction was collected by UV monitoring, chilled ethanol (manufactured by Wako Pure Chemical Co., Ltd., 2.5 times the volume of the collected DNA fraction) and 3 M sodium acetate (pH 5.2, one-tenth the volume of the collected DNA fraction) were added to precipitate DNA.

After the precipitated DNA was separated and recovered by centrifugation, it was dissolved in 100 μl of sterile water. To 90 μl of a sample, 11 μl of 2 N sodium hydroxide was added and a treatment was carried out for 10 minutes, whereby cytosine in the sample DNA was deaminated and converted to uracil.

After the treatment, 30 μl of 3 M sodium acetate (pH 5.2), 70 μl of sterile water and 500 μl of chilled ethanol (manufactured by Wako Pure Chemical Co., Ltd.) were added to the solution, and the mixture was left at −20° C. for 1 hour. The precipitated DNA was recovered, and it was dissolved in 40 μl of sterile water. To 30 μl of the DNA solution, 1.5 μl of a reaction buffer (0.1 M magnesium chloride, 0.2 M Tris-HCl (pH 8)) and 10 μg of DNase I (manufactured by Roche Co.) were added, and a treatment was carried out at 37° C. for 2 hours. Then, 0.4 units of snake venom phosphodiesterase (manufactured by Worthington Co. Ltd.) was added, and further reaction was carried out for 90 minutes. Subsequently, 0.2 units of phosphodiesterase and 2 units of alkali phosphatase (manufactured by Promega Inc.) were added and a treatment was carried out for 90 minutes, whereby DNA was digested into nucleosides. The digested products were separated from proteins or unreacted substances by an operation of ethanol precipitation, and then the solution was dried by suction. After the dried product was dissolved in 30 μl of sterile water, the amount of nucleosides were measured by the foregoing method of HPLC analysis described in 1-D.

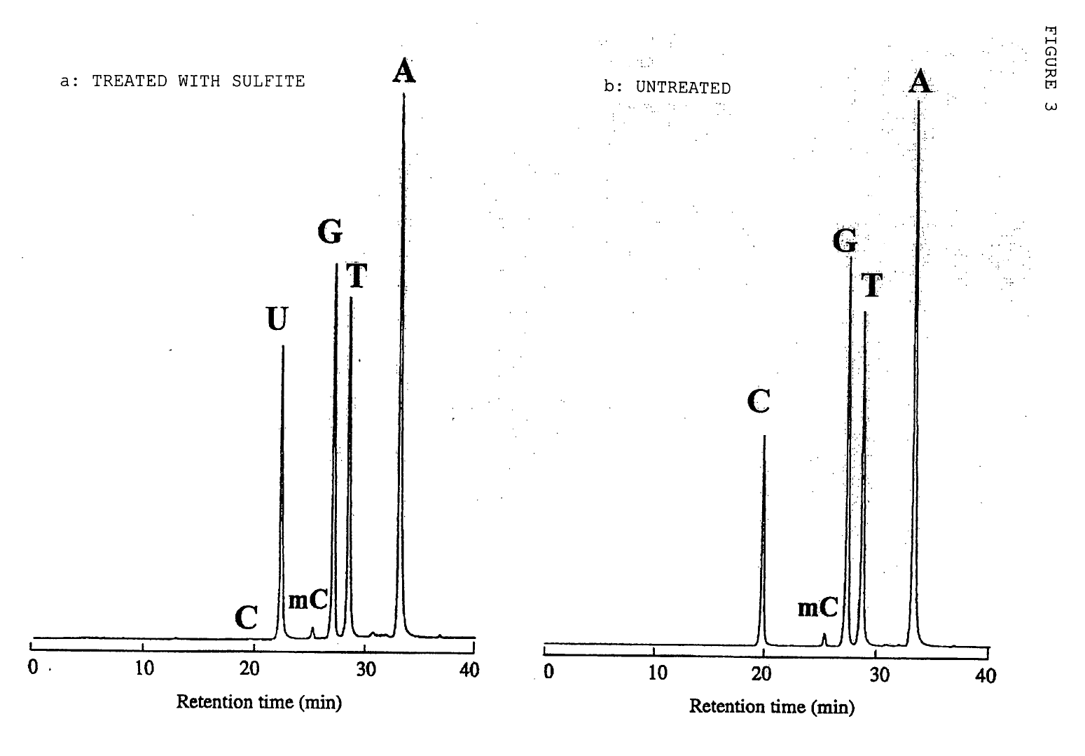

The charts of the HPLC analyses are shown in FIG. 3, and the ratio of each nucleoside is shown in Table 2.

TABLE 2

| | Mol % | | | | | |
|---|---|---|---|---|---|---|
| | C | U | mC | G | T | A |
| Treated with sulfite | 0.08 | 19.89 | 1.29 | 21.56 | 29.28 | 27.90 |
| Untreated | 20.26 | 0.04 | 1.41 | 22.43 | 28.67 | 27.19 |

In Table 2, C indicates 2′-deoxycytidine, U indicates 2′-deoxyuridine, mC indicates 5-methyl-2′-deoxycytidine, G indicates 2′-deoxyguanosine, T indicates thymidine and A indicates 2′-deoxyadenosine.

In 9 M ammonium sulfite-sodium solution, the deamination ratio of cytosine (conversion ratio from citosine to uracil) in genomic DNA when a treatment was carried out at 90° C. for 10 minutes was 99.6%. In addition, the conversion ratio of 5-methylcitosine was 10% or less. Moreover, the conversion of another base was not observed. The reaction times in which the similar deamination ratio was obtained at 70° C. and 37° C. were 16 minutes and 170 minutes, respectively.

### Example 3

### Investigation whether DNA Treated with 9 M Bisulfite Composition is Used as Template

pUC119 (manufactured by Takara Bio Inc.) treated with 1 μg of ScaI restriction enzyme (manufactured by NEB Inc.) was denatured into single-stranded DNAs by treating it in 50 μl of 0.3 N sodium hydroxide solution at 37° C. for 30 minutes. To the treated solution, 500 μl of 10 M ammonium-sodium sulfite solution (pH 5.4) was added and mixed well. Then, a mineral oil was overlaid, and reaction was carried out at 70° C. or 90° C. for 5 minutes to 40 minutes. The reaction solution (130 μl) was taken out and mixed with an equivalent amount of ice-cold sterile water. DNA was purified using Wizard DNA Clean-UP system (manufactured by Promega Inc.) in accordance with the operation manual and dissolved in 90 μl of sterile water. Thereto was added 11 μl of 2 N sodium hydroxide solution, and a treatment was carried out at 37° C. for 10 minutes. By using 10 μg of yeast tRNA (manufactured by Sigma Co., Ltd.) as a carrier, DNA was recovered by an operation of ethanol precipitation and dissolved in 100 μl of TE buffer (10 mM Tris-HCl (pH 8.0), 1 mM EDTA).

By using 1 μl of this solution as a sample, PCR was performed using 2 types of primers shown in SEQ ID Nos. 1 and 2 of the sequence listing and AmpliTaq DNA polymerase (manufactured by Applied Biosystems Inc.) in a 50 μl reaction system. The cycle condition was 95° C. for 3 minutes followed by 30 cycles of 95° C. for 30 seconds, 57° C. for 30 seconds and 70° C. for 3 minutes. Other conditions were in accordance with the operation manual. After the PCR, 1 μl of the sample was analyzed by agarose gel electrophoresis, and the amount of amplification was confirmed.

As a result, the amounts of amplification by PCR of untreated DNA and the DNA of the sample treated at 70° C. or 90° C. for 5 minutes to 40 minutes were almost equal. This suggests that the DNA treated with sulfite was not damaged such as cleaved, in such a manner that the DNA cannot be used as a template for PCR. Further, with regard to the PCR products of the sample treated at 70° C. for 20 minutes and the sample treated at 90° C. for 10 minutes, the nucleotide sequence was determined using BigDye™ Terminator Cycle Sequencing kit (manufactured by Applied Biosystems Inc.) and the ABI model 3700 autosequencer (manufactured by Applied Biosystems Inc.), and it was found that cytosine was converted to thymine.

### Example 4

### Deamination of High Molecular Weight DNA and Detection of Methylated DNA

In has been reported that CpG island of the CDH1 gene and that of the RASSF1A gene are unmethylated and methylated in MCF-7 cells, respectively (see Koizume et al., NucleicAcids Res., 30, 4770-4780, 2002, Dammann et al., Cancer Res., 61, 3105-3109, 2001, and the like). Therefore, it was investigated whether the methylation status of these CpG islands are reproduced after a treatment with 9 M sulfite composition.

4-A. Preparation of Sulfite Treated MCF-7 Genome DNA

The genomic DNA obtained from human breast cancer cells, MCF-7 cells, was digested with a restriction endonuclease, TSP509I. Phenol/chloroform treatment and ethanol precipitation treatment were carried out, and after being dried, DNA was dissolved in 45 μl of sterile water. Then, thereto was added 5 μl of 3 N sodium hydroxide, a treatment was carried out at 37° C. for 30 minutes, whereby DNA was denatured into single-stranded DNAs. To the solution of denatured single-stranded DNAs, either 565 μl (90° C.) or 545 μl (70° C.) of 10 M sulfite composition was added, and a treatment was carried out at 90° C. for 20 minutes or at 70° C. for 40 minutes. After the reaction, DNA was purified using Wizard DNA Clean-UP system (manufactured by Promega Inc.) in accordance with the operation manual and dissolved in 90 μl of sterile water. Thereto was added 11 μl of 2 N sodium hydroxide solution, and a treatment was carried out at 37° C. for 10 minutes. By using 10 μg of yeast tRNA (manufactured by Sigma Co., Ltd.) as a carrier, DNA was recovered by an operation of ethanol precipitation and dissolved in 16 μl of TE buffer (10 mM Tris-HCl (pH 7.5), 1 mM EDTA).

For comparison, a treatment was carried out also by a conventional method. That is, 4 μg of MCF-7 DNA digested in the same manner as described above was treated in 50 μl of 0.3 N sodium hydroxide solution at 37° C. for 30 minutes, thereby denaturing it into single strands. Then, the reaction solution was mixed with 500 μl of 4 M sodium sulfite/1 mM hydroquinone solution, a mineral oil was overlaid, and a treatment was carried out at 55° C. for 20 hours in dark. After the reaction, DNA was purified using Wizard DNA Clean-UP system (manufactured by Promega Inc.) in accordance with the operation manual and dissolved in 90 μl of sterile water. Thereto was added 11 μl of 2 N sodium hydroxide solution, and a treatment was carried out at 37° C. for 10 minutes. By using 10 μg of yeast tRNA (manufactured by Sigma Co., Ltd.) as a carrier, DNA was recovered by an operation of ethanol precipitation and dissolved in 16 ∞l of TE buffer (10 mM Tris-HCl (pH 7.5), 1 mM EDTA).

4-B. Analysis of CDH1 Gene in MCF-7 Cell

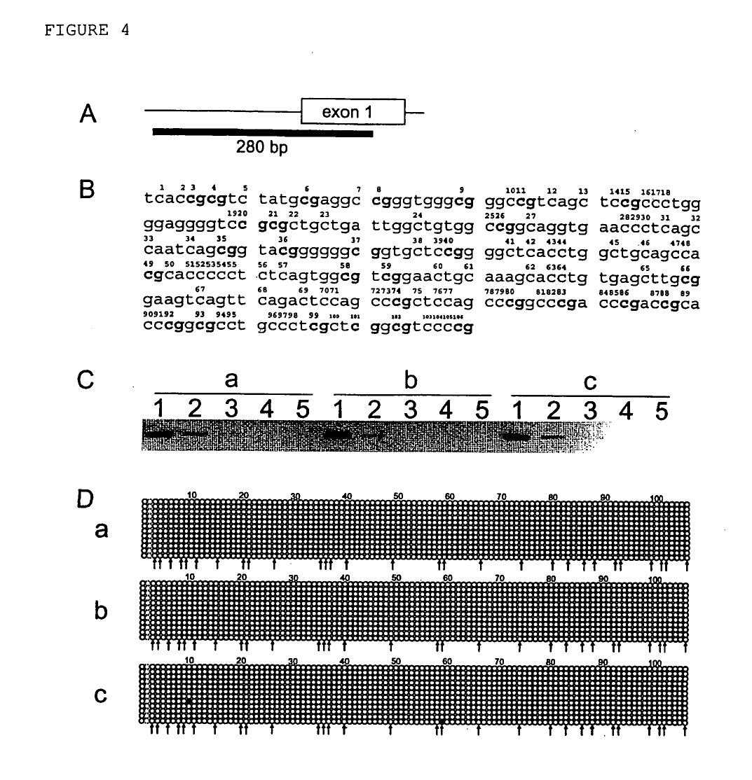

First, methylation status of CDH1 gene in MCF-7 cell was analyzed. By using a sample treated with a sulfite composition as a template, the sequence of a 280-base pair fragment shown in FIG. 4B (or SEQ ID No. 3of the sequence listing) was amplified. PCR analysis was performed in the following procedure.

(PCR Analysis)

MCF-7 DNA treated with sulfite composition was serially diluted with TE (10 mM Tris-HCl (pH 7.5)/1 mM EDTA (pH 8.0)) containing 1.25 mg/ml of yeast tRNA. After the mixture was incubated at 95° C. for 3 minutes, AmpliTaq DNA polymerase Stoffel fragment was added, and 20 cycles (95° C. for 30 seconds, 55° C. for 30 seconds and 72° C. for 30 seconds) of the initial amplification was performed. The reaction was performed in accordance with the literature by Koizume et al (Nucleic Acids Res., 30, pp. 4770-4780, (2002)). Template DNA was used in an amount of 500 ng, 50 ng, 5 ng or 500 pg. In addition, as a PCR primer, the sequences shown in SEQ ID No. 4 (CDH1-L1) and SEQ ID No. 5 (CDH1-R1) of Table 3 or the sequence listing were used. Subsequently, a semi-nested PCR was performed under the same conditions as described above except for performing 30 cycles using 2 μl of the initial PCR reaction solution and the sequences shown in SEQ ID No. 6 (CDH1-L2) and SEQ ID No. 7 (CDH1-R2) of Table 3 or the sequence listing as a PCR primer.

TABLE 3

| Gene | Name of primers | Sequence (5′ to 3′)^{a} | Positions (accession no.) |
|---|---|---|---|
| CDH1 | CDH1-L1 | ATTTAGTGGAATTAGAATAGTGTAGGTTTT | (791-820, L34545) |
| | CDH1-R1 | CTACAACTCCAAAAACCCATAACTAAC | (1139-1165, L34545) |
| | CDH1-L2 | cggaattcTTAGTAATTTTAGGTTAGAGGG | (837-858, L34545) |
| | CDH1-R2 | cgggatcCTACAACTCCAAAAACCCATAACTAAC | (1139-1165, L34545) |
| RASSF1A | RASSF1A-L1 | cggaattcGTTTTGGTAGTTTAATGAGTTTAGGTTTTTT | (18092-18122, AC002481) |
| | RASSF1A-R1 | ACCCTCTTCCTCTAACACAATAAAACTAACC | (17741-17771, AC002481) |
| | RASSF1A-R2 | cgggatCCCCACAATCCCTACACCCAAAT | (17918-17940, AC002481) |

^{a}Lower cases indicate sequences introduced for restriction endonucleases.

First, it was investigated how much DNA could be used as a template. In both cases where MCF-7 DNA treated by the conventional method was used as a template (FIG. 4C, a, Lane 2) and where MCF-7 DNA treated with 9 M bisulfite composition at 90° C. for 20 minutes (FIG. 4C, b, Lane 2) or at 70° C. for 40 minutes (FIG. 4C, c, Lane 2) was used, PCR product was clearly detected when 50 ng of DNA was applied.

Subsequently, the PCR product obtained by using 500 ng of DNA as a template in each experiment was cloned. Twelve plasmid clones were picked up and subjected to nucleotide sequence analysis. The analyzed strand contained 106 cytosine residues in the amplified region, 29 of which were located at CpG sites.

In the case of MCF-7 DNA treated by the conventional method, all cytosine residues were converted to uracil in 12 plasmid clones that were analyzed. In the case where MCF-7 DNA treated with 9 M sulfite composition at 90° C. for 20 minutes or at 70° C. for 40 minutes was used as a template, almost the same results were obtained.

These results suggest that treatment of human genomic DNA with 9 M sulfite composition at a high temperature permits rapid conversion of cytosine to uracil.

4-C. Analysis of RASSF1A Gene in MCF-7 Cell

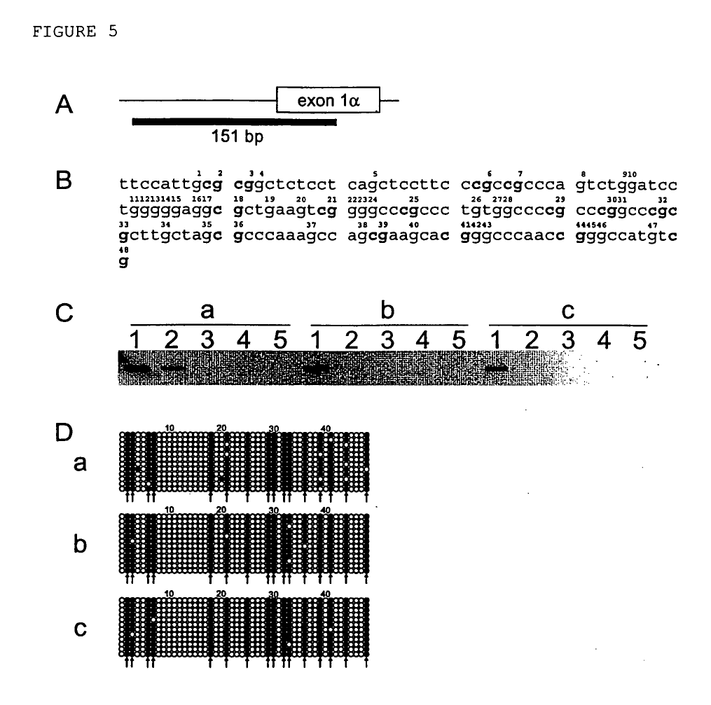

Subsequently, methylation status of the CpG island of RASSF1A gene in MCF-7 cell was analyzed. The sequence of a 151-base pair fragment shown in FIG. 5B (or SEQ ID No. 8 of the sequence listing) was amplified by using a sample treated with a sulfite composition as a template.

(PCR Analysis)

PCR analysis was performed by the same method as in 4-B described above except for the following points. As the initial PCR primer, the sequences shown in SEQ ID No. 9 (RASSF1A-L1) and SEQ ID No. 10 (RASSF1A-R1) of Table 3 or the sequence listing were used. In the semi-nested PCR, 6 μl of the initial PCR reaction solution was used. As the PCR primer, the sequences shown in SEQ ID No. 9 (RASSF1A-L1) and SEQ ID No. 11 (RASSF1A-R2) of Table 3 or the sequence listing were used.

(Measurement Results)

In the case where MCF-7 DNA treated by the conventional method was used as a template, PCR product was detected when 50 ng of DNA was applied (FIG. 5C, a, Lane 2). On the other hand, in the case where MCF-7 DNA treated with 9 M sulfite composition at 90° C. for 20 minutes or at 70° C. for 40 minutes was used as a template, PCR product was detected only when 500 ng of DNA was applied.

These results suggest that the mode of DNA degradation caused by treatment with 9 M sulfite composition at a high temperature varies depending on nucleotide sequences.

The analyzed strand contained 48 cytosine residues in the amplified region, 16 of which were located at CpG sites. When MCF-7 DNA was treated by the conventional method, almost all cytosine residues were converted to uracil at non-CpG sites in all 12 plasmid clones that were analyzed. In contrary, most cytosine residues at CpG sites were not converted. In the case where MCF-7 DNA treated with 9 M sulfite composition at 90° C. for 20 minutes or at 70° C. for 40 minutes was used as a template, similar results were obtained.

As is clear from the results of the Examples described above, it was found that by treating genomic DNA with a sulfite composition having a high sulfite concentration of the present invention, cytosine was converted to uracil in a short time, while most 5-methylcytosine residues were not changed.

Conventionally, for a treatment of converting cytosine to uracil, a long time treatment (about 12 to 16 hours) was required. However, it was found that according to the present invention, conversion of cytosine to uracil can be performed in a short time, and moreover, detection of methylated cytosine can be also rapidly performed.

1. A sulfite composition having a sulfite concentration of more than 6.2 M.

2. The sulfite composition according to claim 1 having a sulfite concentration of more than 6.2 M and 10 M or less.

3. The sulfite composition according to claim 1 having a pH of 5.0 to 5.6.

4. The sulfite composition according to claim 1 comprising 2 types or more of sulfites.

5. The sulfite composition according to claim 1 comprising 2 types or more of sulfites selected from the group consisting of ammonium salts and sodium salts of sulfites.

6. The sulfite composition according to claim 1 comprising ammonium sulfite, ammonium bisulfite and sodium bisulfite.

7. A method for deaminating DNA comprising the following steps of:
(1) treating a sample containing a single-stranded DNA with a sulfite composition having a sulfite concentration of more than 6.2 M; and
(2) treating the sample treated in (1) with an alkali.

8. The method for deaminating DNA according to claim 7 comprising the following step (0) before the step (1):
(0) denaturing a double-stranded DNA in the sample into single-stranded DNAs.

9. The method for deaminating DNA according to claim 7, wherein the DNA in the step (1) is DNA comprises cytosine.

10. The method for deaminating DNA according to claim 7, wherein the sulfite composition in the step (1) is a sulfite composition having a sulfite concentration of more than 6.2 M and 10 M or less.

11. The method for deaminating DNA according to claim 7, wherein the step (1) is a step of performing the treatment in a pH range of about 5 to 5.6.

12. The method for deaminating DNA according to claim 7, wherein the step (1) is a step of performing the treatment at a temperature of about 60 to 95° C. for about 5 to 60 minutes.

13. A method for detecting methylated DNA comprising the following steps of:
(a) performing deamination treatment by treating a sample containing a single-stranded DNA with a sulfite composition having a sulfite concentration of more than 6.2 M and treating it with an alkali; and
(b) detecting methylated DNA in the sample obtained in (a).

14. The method for detecting methylated DNA according to claim 13, wherein the DNA in the step (a) is DNA comprises cytosine, and the step (b) is a step of detecting methylated cytosine in the sample obtained in (a).

15. The method for detecting methylated DNA according to claim 14, wherein the step (b) is a step of detecting methylated cytosine in the sample by using any of nucleotide sequence determination, a DNA chip and a restriction enzyme.

16. The method for detecting methylated DNA according to claim 14, wherein the step (b) is a step of detecting methylated cytosine by means of amplifying DNA in the sample using at least one primer that can amplify a nucleic acid in the case where cytosine of DNA is converted to uracil and at least one primer that can amplify a nucleic acid in the case where cytosine is not converted to uracil, and identifying the locations of 5-methylcytosine and uracil based on the presence or absence of amplification.

17. A kit for deaminating DNA comprising a sulfite composition according to claim 1.

18. A kit for detecting methylated DNA comprising a sulfite composition according to claim 1.


SEQUENCE LISTING (11 sequences)

SEQ ID NO: 1 - DNA, 26 bp, artificial
  primer
cggaattctattggttaaaaaatgag

SEQ ID NO: 2 - DNA, 27 bp, artificial
  primer
aactgcagacattaacctataaaaata

SEQ ID NO: 3 - DNA, 280 bp, human
tcaccgcgtctatgcgaggccgggtgggcgggccgtcagctccgccctggggaggggtccgcgctgctgattggctgtggccggcaggtgaaccctcagccaatcagcggtacggggggcggtgctccggggctcacctggctgcagccacgcaccccctctcagtggcgtcggaactgcaaagcacctgtgagcttgcggaagtcagttcagactccagcccgctccagcccggcccgacccgaccgcacccggcgcctgccctcgctcggcgtccccg

SEQ ID NO: 4 - DNA, 30 bp, artificial
  primer
atttagtggaattagaatagtgtaggtttt

SEQ ID NO: 5 - DNA, 27 bp, artificial
  primer
ctacaactccaaaaacccataactaac

SEQ ID NO: 6 - DNA, 30 bp, artificial
  primer
cggaattcttagtaattttaggttagaggg

SEQ ID NO: 7 - DNA, 34 bp, artificial
  primer
cgggatcctacaactccaaaaacccataactaac

SEQ ID NO: 8 - DNA, 151 bp, human
ttccattgcgcggctctcctcagctccttcccgccgcccagtctggatcctgggggaggcgctgaagtcggggcccgccctgtggccccgcccggcccgcgcttgctagcgcccaaagccagcgaagcacgggcccaaccgggccatgtcg

SEQ ID NO: 9 - DNA, 39 bp, artificial
  primer
cggaattcgttttggtagtttaatgagtttaggtttttt

SEQ ID NO: 10 - DNA, 31 bp, artificial
  primer
accctcttcctctaacacaataaaactaacc

SEQ ID NO: 11 - DNA, 29 bp, artificial
  primer
cgggatccccacaatccctacacccaaat